(19) United States
(12) Patent Application Publication
(10) Pub. No.: US 2006/0234215 A1
(43) Pub. Date: Oct. 19, 2006

(54) Rapid exchange luminescence (REL) for high sensitivity detection

(76) Inventor: Bruce S. Hudson, Syracuse, NY (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/406,119

(22) Filed: Apr. 18, 2006

Related U.S. Application Data
(60) Provisional application No. 60/672,492, filed on Apr. 19, 2005.

(51) Int. Cl. C12Q 1/70; C12Q 1/68

(52) U.S. Cl. 435/5; 435/6; 977/924

(57) ABSTRACT

A bioswitch is described which includes a long-lived emitter such as a lanthanide luminophore for time gated detection of ligand binding without interfering background signal.

### CROSS-REFERENCE TO RELATED APPLICATIONS

This application claims priority to provisional application No. 60/672,492, filed Apr. 19, 2005 which is incorporated herein by reference.

### BACKGROUND OF THE INVENTION

1. Field of the Invention

This invention discloses a general method for the detection of analytes in aqueous solution using a luminescent sensor. It is applicable to a wide variety of analytes ranging from metal ions to pathogenic organisms. The physical basis of this invention is rapid exchange between two chemical states on a time scale that is faster than the emission of light following pulsed excitation. The implementation of this invention based on sensitized terbium luminescence is described.

2. Description of the Related Art

Fluorescent or luminescent sensor applications are based on an equilibrium S+A [symbol] S*A where a non-emissive sensor form, S, is converted to an emissive sensor form, S*, on binding to an analyte, A. Measurement of the emission signal permits determination of the concentration of analyte A. In such cases the detection limit for A is limited by the equilibrium process S [symbol] S* which contributes a background signal in the absence of A. Decreasing the equilibrium constant for the S [symbol] S* reaction so as to reduce the background level also reduces the affinity of the sensor for the analyte and is thus not a useful solution for high sensitivity detection.

Time-gated detection, the integration of emission signal after a time delay following pulsed excitation, is a very efficient way to eliminate stray excitation light, Raman scattering and adventitious fluorescence. Long lived sensor species are particularly useful in this regard because the delay can be set to a larger value to more efficiently reject background without loss of signal. This is particularly useful in analytical applications that involve environmental samples that may contain fluorescent materials. Time-gated detection is not particularly helpful in removing the background from S*, however, because S* and the analyte complex S*A will have very similar lifetimes at least in those cases where A is a microorganism or protein.

Sensitized terbium (Tb⁺³) luminescence has become a very valuable tool in biotechnology applications (Johansson, M. K., et al. Time Gating Improves Sensitivity in Energy Transfer Assays with Terbium Chelate/Dark Quencher Oligonucleotide Probes. J. Am. Chem. Soc. 2004, 126, 16451-16455; Choppin, G. R, et al., Applications of lanthanide luminescence spectroscopy solution studies of coordination chemistry. Coordination Chemistry Reviews, 1998, 174, 283-299; Bunzli, J-C. G. Chapter 7 Luminescent Probes. Lanthanide Probes in Life, Chemical and Earth Sciences Theory and Practice, Bunzli, J.-C., G; Choppin, G. R. Eds. Elsevier, New York, 1989. p. 219-293). The utility of sensitized Tb⁺³ luminescence derives primarily from its long lifetime (ca. 1 ms) permitting easy time-gated detection. Sensitization of the excitation process via energy transfer from a chromophore is needed for such applications in order to overcome the extremely low extinction coefficient of the ion itself.

Sensitized terbium functions in time-resolved fluorescence resonance energy transfer (TR-FRET) by transferring energy to a nearby acceptor molecule, usually a fluorescent acceptor such as rhodamine or fluorescein. The transferred energy can be detected as a fluorescence signal.

While the excited state lifetimes of the fluorescent acceptors are on a nanosecond time scale, the excited-state lifetime of a terbium chelate is on a millisecond time scale. Time-resolved detection techniques on this time scale are easily and inexpensively implemented. By waiting 100 microseconds after excitation, interfering fluorescence from other assay components, including direct excitation of the acceptor fluorophore, can be gated out. This provides a high (several orders of magnitude higher) signal-to-background ratio for detection of a species such as terbium with a long lifetime.

This technology has not been applied to molecular switches which typically employ a fluorescent entity and a quencher, configured so that there is a change in the signal from the fluorophore upon binding of a target ligand. Placing a lanthanide chelate with a long excited state lifetime in proximity to a fluorophore capable of energy transfer over long distances has posed a problem in efficient fluorescence quenching in a relatively small molecule such as an oligonucleotide construct. Consequently, it has not been possible to take advantage of the high signal to background ratio possible using lanthanide chelates in a molecular switch. Embodiments of the invention are directed to the use of lanthanide chelates and other long lifetime luminophores which overcome the aforesaid problems.

### SUMMARY OF THE INVENTION

Embodiments of the invention are directed to a molecular switch, which includes a binding domain for a ligand, a framework and a signaling apparatus. The signaling apparatus has a long-lived emitter molecule and short range quencher molecule located along the framework with changeable positions relative to one another. A difference is detectable in a fluorescent signal upon change in conformation between two predominantly populated conformational states of the switch. One conformational state binds the ligand and the other conformational state does not, and there is interchange between these two conformational states that is rapid compared to the emission lifetime of the long-lived emitter.

In preferred embodiments, the switch includes a nucleic acid and/or one or more modified nucleotide monomers. More preferably, the nucleic acid has a double-hairpin construct.

In preferred embodiments, the short range quencher is a quencher based upon electron transfer processes. More preferably, the quencher is a nitroxide. In a most preferred embodiment, the nitroxide is TEMPOL or a derivative thereof.

In preferred embodiments, the long lived emitter molecule is a lanthanide chelate, a ruthenium chelate or a rhenium chelate. In a most preferred embodiment, the long-lived emitter is a lanthanide chelate which is CS124-DTPA. In some preferred embodiments, the long lived emitter has a emission lifetime of 10 μsec to 10 msec. In other preferred embodiments, the long lived emitter has an emission lifetime of 0.1 to 300 μsec.

In some preferred embodiments, the ligand is ricin, cryptosporidium or its oocysts, giardia or its cysts, E. coli, Shiga-like toxin producing E. coli O157:H7 strain, Legionella Pneumophila, or Staphylococcus aureus.

In some preferred embodiments, the ligand is involved in the etiology of a viral infection, which is selected from Hepatitis C, Congo-Crimean hemorrhagic fever, Ebola hemorrhagic fever, Herpes, human cytomegalovirus, human pappiloma virus, influenza, Marburg, Q fever, Rift valley fever, Smallpox, Venezuelan equine encephalitis, HIV-1, MMTV, HIV-2, HTLV-1, SNV, BIV, BLV, EIAV, FIV, MMPV, Mo-MLV, Mo-MSV, M-PMV, RSV, SIV, and AMV.

In preferred embodiments, the ligand is TAR-tat, RRE-rev, DIS, PBS, RT, PR, IN, SU, TM, vpu, vif, vpr, nef, mos, tax, rex, sag, v-src, v-myc and precursors and protease products of the precursors, gag, gag-pol, env, src, or onc.

In preferred embodiments, the ligand is derived from an organism which is selected from bacteria, fungi, insects, and pathogens and pests to humans, animals, and plants.

In preferred embodiments, the ligand is a toxin or other factor derived from bacteria and other microorganisms selected from B. anthracis, Burkholderia pseudomallei, Botulinum, Brucellosis, Candida albicans, Cholera, Clostridium perfringins, Kinetoplasts, Malaria, Mycobacteria, Plague, Pneumocystis, Schistosomal parasites, Cryptosporidium, Giardia, and other environmental contaminants of public and private water supplies, Ricin, Saxitoxin, Shiga Toxin from certain strains of E. coli, Staphylococcus (including enterotoxin B), Trichothecene mycotoxins, Tularemia, and agents causing Toxoplasmosis, and food or beverage contaminants that may be deleterious to human or animal health.

In preferred embodiments, the ligand is a small-molecule target such as nerve gas agents, chemical poisons, contaminants of public and private water supplies, food and beverage contaminants, and contaminants of indoor air that may be deleterious to human or animal health.

Embodiments of the invention are directed to a diagnostic method for detecting the presence of a ligand molecule in a sample, which includes one or more of the following steps.

1) providing a molecular switch as described above;

2) contacting the molecular switch with the sample;

3) pulsing the molecular switch with an excitation pulse of an appropriate first wavelength;

4) delaying measurement of the emission spectra for 0.1 μsec to 1 msec; and

5) measuring the emission spectra at an appropriate second wavelength to determine the presence of the ligand molecule.

In preferred embodiments, the switch includes a chimeric DNA-RNA molecule and/or one or more modified nucleotide monomers.

Preferably, the ligand is an infectious organism or toxic agent. More preferably, the method is adapted for use in a field kit for real-time detection of the infectious organism or toxic agent.

In preferred embodiments, the excitation pulse is for 1-20 ns. In some preferred embodiments, measurement of the emission spectra is delayed for 10 to 500 μsec. In alternate preferred embodiments, measurement of the emission spectra is delayed for 0.1 to 10 μsec.

In a most preferred embodiment, the luminophore is CS124-DTPA, and the first wavelength is 340 nm with a 30 nm bandpass.

Embodiments of the invention are directed to an assay method for discovering a chemical entity that interferes with ligand binding, which includes one or more of the following steps.

(a) providing a molecular switch as described above;

(b) contacting the molecular switch with a ligand in the absence of the chemical entity;

(c) pulsing the molecular switch with an excitation pulse of an appropriate first wavelength;

(d) delaying measurement of the emission spectra for 0.1 μsec to 1 msec;

(e) measuring the emission spectra at an appropriate second wavelength to determine the presence of the ligand molecule, and monitoring the signal;

(f) contacting said molecular switch with said ligand in the presence of the chemical entity;

(g) repeating steps (c)-(e) to determine the binding of the ligand in the presence of the chemical entity; and

(h) comparing the signals generated in the presence and absence of the chemical entity to determine whether the chemical entity interfered with the binding of the ligand.

In preferred embodiments, the switch includes one or more modified nucleotide monomers. In preferred embodiments, the ligand is a viral protein.

In preferred embodiments, the step of contacting the molecular switch with the ligand in the presence of the chemical entity, also includes allowing the molecular switch and the ligand to equilibrate prior to adding the chemical entity. More preferably, the molecular switch is adapted to generate a null fluorescent signal upon equilibration with the ligand.

In some preferred embodiments, the binding domain includes a combinatorially-derived sequence which has been empirically chosen to bind the ligand.

In some preferred embodiments, the measurement of the emission spectra is delayed for 10 to 500 μsec. In alternate preferred embodiments, the measurement of the emission spectra is delayed for 0.1 to 10 μsec.

Further aspects, features and advantages of this invention will become apparent from the detailed description of the preferred embodiments which follow.

### BRIEF DESCRIPTION OF THE DRAWINGS

These and other feature of this invention will now be described with reference to the drawings of preferred embodiments which are intended to illustrate and not to limit the invention.

FIG. 1. The cs-124 DTPA terbium ion complex. The efficiency of energy transfer from the carbostyryl 124 dye to the terbium ion is ca. 0.3.

FIG. 2. Illustration of energy transfer from terbium to rhodamine.

FIG. 3. Structure of TEMPO.

FIG. 4. Diagram of the Tb⁺³ cs124-DTPA complex and a TEMPO derivative attached to 3′ and 5′ ends of DNA strands.

FIG. 5. A ricin OrthoSwitch (top) and its corresponding chemical equilibrium (bottom).

### DETAILED DESCRIPTION OF THE PREFERRED EMBODIMENT

While the described embodiment represents the preferred embodiment of the present invention, it is to be understood that modifications will occur to those skilled in the art without departing from the spirit of the invention. The scope of the invention is therefore to be determined solely by the appended claims.

All of the references cited in this application are expressly incorporated herein by reference thereto. Any technical terms and abbreviations, not explicitly defined below, are to be construed in accordance with their ordinary meaning as understood by one of skill in the art of molecular biology. For example, A, C, G, T and U are standard one-letter symbols for the nucleotide bases, adenine, cytosine, guanine, thymine and uracil, respectively. The following specific abbreviations are used in this application:

Definitions

“Orthoswitch”, “Bioswitch”, “Molecular Switch” or “Designed Sensor Construct” means a construct that provides a signal upon binding of a ligand. For example, the signal may be the quenching of a fluorescent signal caused by a conformational change in the sensor construct upon binding a ligand. Conversely, the signal of the orthoswitch may be quenched in the unbound state and upon ligand binding, the quencher may be moved distal to the fluorophore so that a signal is then detected.

“Combimers” refer to nucleic acid constructs that have binding affinity for a target. We define “combimers” to be high affinity combining sequences in a secondary structure context that ensures availability of the binding sequence for binding to the target. By definition, the combimer includes the full secondary structure of the species identified as having affinity for a particular target. An Aptamer is one type of combimer, derived by in vitro evolution (Ellington, A. D., et al. (1990) Nature, 346, 818-822) or the similar SELEX method (Tuerk, C., et al. (1990) Science, 249, 505-510). An Aptamer is a nucleic acid sequence that shares high binding affinity with a Combimer but does not have a predetermined secondary structure.

“Lanthanide chelator” is used to describe a group that is capable of forming a high affinity complex with lanthanide cations such as Tb⁺³, Eu⁺³, Sm⁺³, Dy⁺³. Any fluorescent lanthanide metal can be used in the chelates of this invention but it is expected that chelates containing europium or terbium will possess the best fluorescent properties.

“Luminescence,” “luminescent,” and “luminiophore” are used to distinguish long-lived “fluorescence,” “fluorescent” species or “fluorophores,” respectively. Occasional reference will be made to lanthanide fluorescence, etc. This still refers to long-lifetime emission and is not meant to convey any difference from lanthanide luminescence.

“Oligonucleotide” refers to a nucleotide sequence containing DNA, RNA or a combination. An oligonucleotide may have any number of nucleotides theoretically but preferably 2-200 nucleotides, more preferably 10-100 nucleotides, and yet more preferably 20-40 nucleotides. The oligonucleotide may be chemically or enzymatically modified.

“Target”, “Analyte” or “Ligand” means the putative binding partner for the combimers section of the bioswitch and includes but is not limited to polymers, carbohydrates, polysaccharides, proteins, peptides, glycoproteins, hormones, receptors, antigens, antibodies, DNA, RNA, organisms, organelles, small molecules such as metabolites, transition state analogs, cofactors, inhibitors, drugs, dyes, nutrients, and growth factors and biological complexes or molecules including those that are toxic.

Combinatorially-derived sequence refers to a nucleic acid molecule adapted to bind to a specific molecular target, such as a protein or metabolite

Embodiments of the invention relate to fluorescent or luminescent sensors for use in bioswitches which interact with a ligand to generate a detectable signal. Preferred embodiments relate to sensors which include lanthanide luminophores which have emission lifetimes on the order of hundreds of microseconds to one msec and can be used in time-gated detection methods. In alternate preferred embodiments, discussed below, an emitting species with a lifetime in the range of a few microseconds are described for some applications. This is exemplified by the emission properties of complexes involving ruthenium (Ru) or rhenium (Re) transition metal ions.

Further the two states of the sensor, S and S* must have an equilibrium, S [symbol] S*, with the property that k=kf+kr (where kf is the forward rate of the reaction and kr is the reverse rate of the reaction) is such that the lifetime of the luminescent species, τ>>1/k, i.e., the luminophores S and S* interchange rapidly compared to the emission lifetime.

Fluorescence probes used in bioswitch applications often utilize FRET or other quenching interactions to provide an on-off signal indicating that the switch has interacted with some target species. Typically, the molecular switch includes an analyte binding domain, a framework and a signaling apparatus, which includes the fluorescent or luminescent sensor and is adapted to generate the signal. The signaling apparatus includes a luminophore and a quencher of the luminophore located along the framework. The molecular switch is adapted to reversibly change from a first conformation (S) to a second conformation (S*) upon binding of the analyte. The S* conformation is stabilized as S*A and a fluorescent signal is detected. The relative positions of the fluorophore and quencher change when the nucleic acid switches between first and second conformations, such that the signal generated by the signaling apparatus produces a detectable change.

An example is a molecular beacon switch consisting of a nucleic acid bearing a fluorophore that is proximal to a quencher group in one stable configuration. Binding to an analyte, A (typically a nucleic acid complementary to the beacon sequence), results in a new extended configuration (S*A) with a longer distance between the fluorophore and quencher and thus in a detectable signal. Such applications depend on the fact that the quenching process has a distance dependence that depends steeply on the donor—acceptor distance. In particular, FRET varies with distance according to the factor (1+(R/R₀)⁶)⁻¹ where R₀ is the distance at which energy transfer is 50% efficient. Conventional fluorescence probes have lifetimes that are on the order of a few nanoseconds.

When the relaxation rate for the conversion of the non-emissive sensor, S, to the emissive sensor form, S*, (S [symbol] S*) interchange process is faster than the luminescence emission, time-gated detection can be used to suppress the unwanted background sensor signal. Typically, a short excitation pulse at an appropriate wavelength is followed by time-resolved detection of the emission spectra. In the rapid exchange limit the decay of a mixture of S and S* will be a single exponential with a lifetime that is the average of that for S and S* weighted by their equilibrium fractions. The S [symbol] S* equilibrium will be strongly in favor of the short lived S form and thus free S* will have a short lifetime since it converts to S before it emits. The complex of the emissive sensor form and the analyte, A, (S*A) will be stable for a time that is longer than the emission time because of the high affinity of S* for A. From the photophysical point of view this is kinetic isolation. Because of the resulting large difference in rate of decay of free S* and that in the S*A complex, time-gated detection can fully suppress the background from the S [symbol] S* equilibrium and thus permit very high sensitivity detection of A. In other words, the background signal from the emissive sensor form can be gated out. All of the signal is then due to the S*A form.

This dynamic effect of emission properties has previously been demonstrated for Eu⁺³ ions where the excitation spectrum of the unsensitized emission of the ion is shifted due to complexation (Horrocks, W. D. Jr., et al. Kinetic Parameters For a System at Equilibrium from the Time Course of Luminescence Emission: A New Probe of Equilibrium Dynamics. Excited-State Europium (III) as a Species Label. J. Am. Chem. Soc, 1983, 3455-3459; Ermolaev, V. L., et al. Novel Spectral-Kinetic Methods for Investigation of Ligand Exchange in Labile Metal Complexes in Solutions. Inorganica Chimica Acta, 1984, 95, 179-185). This involves Eu⁺³(L)ₙ₋₁+L [symbol] Eu⁺³(L)ₙ type equilibria where the lifetimes of the two Eu⁺³ species differ. Slow exchange results in two decays with limiting values, rapid exchange results in a single average decay and intermediate exchange results in two component decays whose amplitudes and lifetimes depend on the exchange rates. These fundamental observations have been applied by the inventor to the analysis of a system that, because it involves sensitized excitation, has potential applications as a sensor species (Sharon A. Rivera and Bruce S. Hudson, “Rapid exchange luminescence: Nitroxide quenching and implications for sensor applications”, J. Am. Chem. Soc. 2006; 128(1); 18-19).

Short-Range Lanthanide Luminescence Quenching for Bioswitch Applications with High Sensitivity and Rapid Exchange Background Suppression

Lanthanide luminescence (especially that of the terbium cation, Tb⁺³) has a lifetime that is on the order hundreds of microseconds to one millisecond. This long lifetime makes it possible to detect the emission from Tb⁺³ and other lanthanides such as Eu⁺³ with extremely high sensitivity using time-gated detection. The use of an initial 10-100 μs “off” gate suppresses all stray light and extraneous fluorescence resulting in extremely low background noise. The apparatus needed to implement time-gated detection in this time range is inexpensive and reliable. In one version, a continuous light source illuminates a flowing sample. The detectors are placed downstream at a distance corresponding to flow arrival times that range from 10 μs to a few ms.

Fluorescence resonance energy transfer from Tb⁺³ to red absorbing fluorophore acceptors occurs over very long distances. The value of R₀ can be 100 Å. This has been used for numerous biophysical applications. However, for bioswitch applications the long range nature of this transfer makes it difficult to arrange structural changes that are large enough that the FRET is turned off in either conformation. For this reason, lanthanide luminophores have not been previously utilized in bioswitch applications.

Embodiments of the invention describe the use of a short-range quenching interaction to modulate the Tb⁺³ luminescence. In preferred embodiments, the fluorophore acceptor (e.g. rhodamine) is replaced by a short range quencher molecule (e.g. TEMPOL). The long range energy transfer is replaced by short range quenching interactions that can be adapted to bioswitch applications.

The same methods apply to other lanthanide luminophores. Embodiments of this invention combine the extreme sensitivity of lanthanide luminescence derived from the ease of time-gated detection to remove background signal with the ability to switch this signal on and off on the basis of target binding. This application is particularly relevant to OrthoSwitches involving bistable nucleic acid structures.

Fluorescence emission usually occurs on a time scale that is short compared to that associated with the interconversion of biopolymer species. Fluorescence data often reveals the presence of multiple conformational species as individual fluorescence decay components or spectrally distinct signals. The luminescence lifetime of Tb⁺³ and other lanthanides occurs in a time scale that is on the order of 10⁵-10⁶-fold slower than conventional fluorescence. This has the consequence that the time scale of the emission is slow compared to many conformational changes of biopolymer species. This time-scale aspect of lanthanide emission can be used to advantage in the design of nucleic acid switches designed to have high sensitivity. Specifically, the small component of long lived “on” form of a switch (S*) that is necessarily in equilibrium with the predominant “off” form in the absence of “target” (S) will be dynamically averaged. This means that this “background” switch signal can be “gated out” along with the other short-lived luminescence.

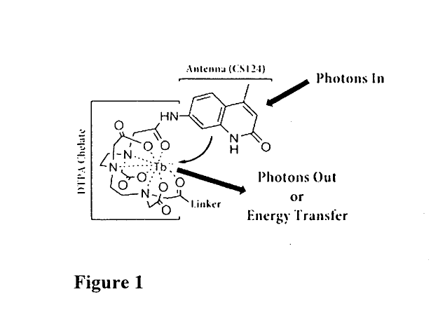

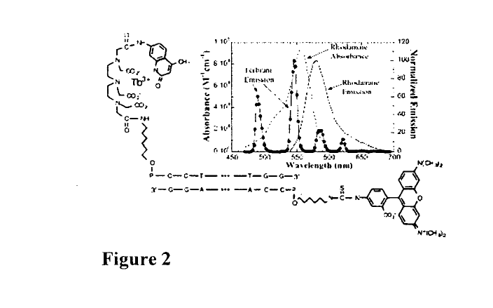

A specific implementation of this concept is based on the use of the chelation-sensitizer complex cs124-DTPA (such as PanVera's LanthaScreen™). Proteins and peptides can be labeled via either the free amino group or exposed cysteine using CS124-DTPA according to the manufacturer's protocol. Nucleic acids such as oligonucleotides may be labeled using an amine modification of the nucleic acid according to the manufacturer's protocol. The prior art teaches that the CS124-DTPA complex binds the Tb⁺³ ion and provides an efficient method for activation of its luminescence via the cs-124 carbostyryl chromophore as shown in FIG. 1. This complex is used as a fluorescence label or as a FRET donor in applications in which it is attached to the macromolecule (i.e., DNA or a protein) and energy transfer is measured to an acceptor species such as rhodamine (FIG. 2; see also PanVera Lit #762-038205)). The attachment of this chelation/activation structure to a protein or nucleic acid uses well-established chemical methods. The combination of the sensitized terbium luminophore as a donor and a chromophoric acceptor is well-suited to long-range energy transfer determinations of the distance between the donor and the acceptor, but not for molecular bioswitch applications.

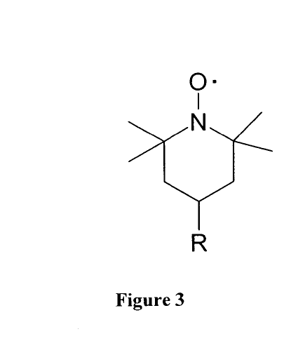

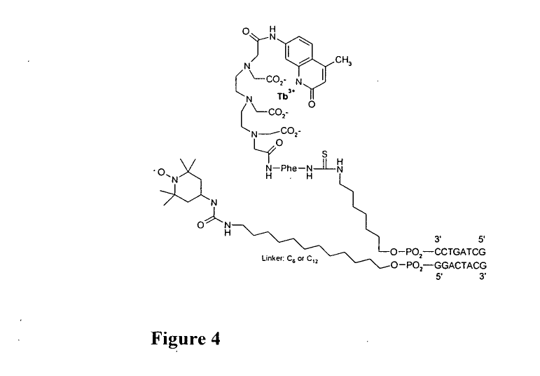

In preferred embodiments of the invention, the rhodamine acceptor species is replaced by a short range quencher such as the nitroxide species TEMPO (R═H in FIG. 3). TEMPO is known to quench the emission of terbium by collisional quenching. The mechanism of this quenching is probably an electron transfer process. Such processes are known to be short range in nature, depending on the overlap of the electronic wavefunctions. Collisional quenching of this type has limited biophysical or biotechnological applications. However, TEMPO derivatives (e.g., R═—NH₂) can be attached to nucleic acids or proteins using well established methods. A specific example of this construct is shown in FIG. 4.

As exemplified in FIG. 4, the terbium chelate is attached to the 3′ end of a double stranded segment of an oligonucleotide by a C₆ or C₁₂ linker. The nitroxide quencher is attached to the 5′ end of the opposite strand by a similar linker. In this conformation, the emission signal from the terbium chelate is quenched by the nitroxide quencher. The emission spectrum of Tb⁺³ is shown in FIG. 2. Typically, emission is measured at 545 nm using a narrow band optical filter to reduce signal from other sources, although emission can be measured at any appropriate emission wavelength as shown in FIG. 2.

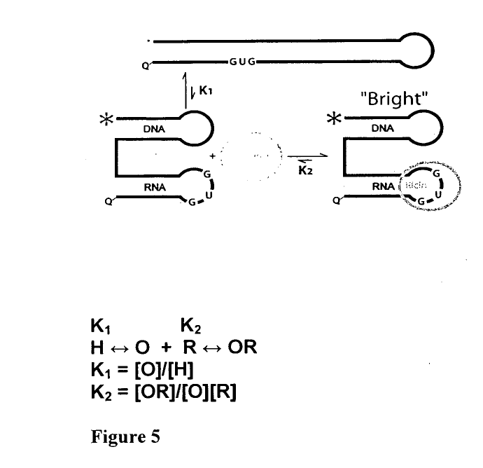

Preferred embodiments of the invention are directed to constructs which include a short-range collisional quencher such as TEMPO in proximity to a sensitized long-lived luminescent species such as a lanthanide chelate phosphor (here Tb⁺³). FIG. 5 shows a schematic bioswitch (OrthoSwitch) according to preferred embodiments of the invention. The OrthoSwitch is a nucleic acid construct (which may be a chimeric DNA/RNA construct and which may contain non-nucleic acid components) that exists in two stable conformational states designated H and O that are in equilibrium. The equilibrium constant for the equilibrium between H and O is K₁. Parallel segments represent hydrogen-bonded double helices. These two forms, H and O, differ in terms of their fluorescence properties. The O form binds to an analyte “target” (ricin or R in this diagram) but the H form does not. In the H form, the quencher (Q) is proximal to the lanthanide chelate (*) and fluorescence is quenched. In the O form or the OR form, Q is too far away from the lanthanide chelate to quench the signal. In preferred embodiments, Q is only capable of short range quenching action. In this case, a long lived fluorescent signal from the terbium is detected in the O and OR forms. The fluorescent signal produced by the unbound O form can be gated out because of the rapid equilibrium between H and O. The average fluorescent lifetime for H and O is much shorter than the fluorescent lifetime of OR. The presence of the analyte results in a change in the fluorescence signal because of a change in the position of the H [symbol] O equilibrium.

In preferred embodiments, three independent factors are combined to create an OrthoSwitch. The first factor is a structure that binds to the analyte in one form but not in another. In this case, the RNA stem-loop structure of 0 binds to ricin while the double helical structure containing this sequence does not. In general this structure is a Combimer, a sequence in a defined secondary structure that has been shown to have high affinity for a particular target species.

The second factor is an H/O pair containing the Combimer with an equilibrium constant K₁=10⁻¹-10⁻⁵. This aspect depends on prior studies of nucleic acid thermodynamics permitting secondary structure analysis with some reliability. In the example of FIG. 5, bulges and mismatches may be introduced to destabilize the secondary structure of the H form (the quenched form which does not bind the analyte). By such modifications, K₁ is set in the optimal range.

The third factor is attachment of a fluorescent group and a quencher to the nucleic acid sequence in such a way that in one form these two groups are sufficiently well separated that the fluorescence is strong whereas in the other form the two are close enough together that quenching occurs. This can be done using fluorescence resonance energy transfer (FRET). This is difficult even with conventional nanosecond fluorophores because of the small size of the OrthoSwitch. FRET is so efficient with lanthanide luminescent species that FRET cannot be turned “off” with constructs of this size. This technical problem in using lanthanide luminophores in bioswitch applications is addressed with the terbium/nitroxide combination described here.

There are several relevant features of embodiments of the invention which address this technical problem. First, the construct of FIG. 4 shows the TEMPO nitroxide attached to a flexible chain linker. The Tb⁺³-cs124-DTPA linker is also relatively long. This makes it possible for the TEMPO group to collide with, and quench, the terbium chelate at some point during the long emission lifetime of Tb⁺³. In essence this is diffusion enhanced quenching. In preferred embodiments, the length of the linker is 4-20 carbon atoms, more preferably, 6-12 carbon atoms.

A second aspect of this technology concerns the effect of the long lifetime of the emission on the sensor background signal. In the absence of target analyte there will be a low level of the “on” state due to the unimolecular equilibrium with constant K₁. This ambient background sets the level that must be matched by conversion of “off” to “on” state by analyte binding. When the bimolecular equilibrium (i.e. R+O [symbol] RO, with the equilibrium constant K₂) increases the level of “on” state so that it is now twice the ambient background then the detector signal (“on” minus background) is equal to the background level. This level, and the value of K₂, set the minimum analyte concentration that can be detected. The background level can be reduced by making K₁smaller. However, this reduces the concentration of “on” form in the bimolecular analyte binding equilibrium and thus results in a proportional decrease in the signal level at low analyte concentration and so has no effect on the analyte concentration that results in a minimal signal.

For steady-state detection of the fluorescence signal the “background” fluorescence due to O and the “signal” fluorescence due to the complex OR are weighted equally. The same is true for a time-gated detection signal when the fluorophore used has a typical nanosecond lifetime. The binding of the target to the O form of the OrthoSwitch results in a change in the concentration of the species in the O form (O plus OR) but does not change the properties of the fluorophore. Thus the emission from O and from OR are indistinguishable spectrally or temporally.

However, without intending to be limited by theory, it is believed that in the case of a long-lived luminescent species like terbium, that the H [symbol] O equilibrium is in rapid exchange on the time scale of the emission. The result of this rapid equilibration is that the luminescence of the emissive species will have a decay constant that is a weighted average of that of the O and H forms. The decay time for the O form is ca. 1 ms. The decay time for the H form will be on the order of 100 times less than that or ca. 10 μs or less. Since the value of K₁ will, by design, favor H over O by 10-100 (K₁=10⁻¹-10⁻²), the decay of the fluorescence of terbium will have a lifetime close to that of the H form of 10 μs.

We now estimate the corresponding situation for the luminescence decay of the complex OR. The value of K₂, the association constant for the target species R with the O form of the switch, will be 10⁹ M⁻¹ or greater (K_{d}=10⁻⁹ or less). The equilibrium constant K₂ is the ratio of the forward rate for complex formation, k_{f}, to the reverse rate, kᵣ, corresponding to its dissociation with K₂=k_{f}/kᵣ. The largest conceivable value of the forward rate is k_{f,max}=10¹⁰ M⁻¹ s⁻¹ which is the diffusion controlled value in aqueous solution. This means that the upper limit for the reverse rate, and thus for the OR [symbol] O [symbol] H exchange rate, is 10 s. The most probable value of k_{f} is 10⁷−10⁸ M⁻¹ s⁻¹ (100-1000 times slower that diffusion controlled) and thus, even if K₂ is only 10⁷ M⁻¹, the off rate will be ca. 1-10 s. This means that the luminescence emission of the Tb⁺³ ion in the OR complex will have a luminescence decay time very near 1 ms.

This long lived emission of the complex is very easy to distinguish from the short lived decay of the H [symbol] O interchange pair. Binding of the target analyte makes the two forms of the long lived emission complex kinetically inequivalent and thus distinguishable. With τ₁=10 μs and τ₂=1 ms, a time-gated detection scheme with an opening delay time of 100 μs enhances the long time contribution to the signal relative to the contribution of the short time component by a factor of 22,000. A 200 μs delay results in a relative suppression of 4×10⁸ with 80% of the long time signal remaining. This feature of this short range quenching of a long lifetime luminescence signal, in combination with suppression of all of the other short lifetime extraneous signals, gives this detection scheme extraordinary detection sensitivity. In preferred embodiments, the time delay is 10 μsec to 1 msec, more preferably, 100 μsec to 500 μsec, yet more preferably, from 150 to 300 μsec. In a most preferred embodiment, a time delay of about 200 μs is used but this depends on K₁ and on the desired sensitivity vs. speed of detection trade-off. That is, a longer time delay provides greater sensitivity. A shorter time delay provides greater speed of detection but some sensitivity is lost. One skilled in the art would know how to choose the appropriate time delay for a given application.

In preferred embodiments, this rapid exchange dynamical averaging scheme depends on the use of a short-range quenching interaction. The use of a nitroxide group as the short range quenching agent is not crucial. In preferred embodiments, the lifetime of the detected species is longer than the interchange time for the two states of the system in the absence of bound target. This is not limited to emission detection but could involve absorption, magnetic resonance or direct electrical signal detection. Binding of the target makes the two states of the switch kinetically inequivalent. Embodiments of the described method allow differentiation between S* (the emissive sensor form) and S*A (the analyte complex) using time-gated detection methods with lanthanide luminophores.

In preferred modes of the molecular switch, the switch is a nucleic acid although the switch can also be a peptide or protein. More preferably, the nucleic acid switch comprises a double-hairpin construct. Yet more preferably, the nucleic acid switch is bistable—i.e., both first and second conformations are stable. In another embodiment, the first and second stable conformations of the switch further comprise double helical and cruciform structures, respectively.

In one mode, the ligand binding domain comprises a naturally-occurring RNA binding site or analog thereof, or a naturally-occurring DNA binding site or analog thereof. Alternatively, the ligand binding domain comprises a combinatorially-derived sequence or related fragment, which is empirically chosen to bind to the ligand.

Any lanthanide chelate phosphor may be used for the bioswitch as described above. Lanthanide chelates typically comprise a chelating group which binds the lanthanide and an organic sensitizer group. The sensitizer group has the function of absorbing light and transferring energy to the lanthanide. It thereby overcomes the inherently low absorbance of the lanthanide ions. Such chelates have been extensively reviewed, for example in Li and Selvin (J. Am. Chem. Soc (1995) 117, 8132-8138).

Lanthanide chelator groups comprising a plurality of polyaminocarboxylate groups are commonly used. European patent EP0203047B1 discloses fluorescent lanthanide chelates comprising “TEKES” (4-(4-isothio-cyanatophenylenthynyl-2,6-{N,N-bis(carboxymethyl)aminomethyl]-pyridine)type photosensitizers. Other suitable examples of chelating groups include those described in WO 96/00901 and WO/99/66780 and in Riehl, J. P. and Muller, G., Handbook on the Physics and Chemistry of Rare Earths, Vol 34, Chapter 220, pages 289-357 (Gschneidner, Jr., K. A.; Bunzli, J-C. G and Pecharsky, V. K, editors, Elsevier B. V., 2005). Preferably the chelating group will be either DTPA (diethylenetriaminepentacetic acid) or TTHA (triethylenetetraaminehexacetic acid). Both DTPA and TTHA are well known in the art and are available from commercial suppliers.

The lanthanide chelator is typically attached to an antenna to absorb light and transfer excitation energy to lanthanide ions. Carbostyril (CS124, 7-amino-4-methyl-2(1 h)-quinolinone and derivatives thereof) are most commonly used (see, for example, Ge, et al. Bioconjugate Chemistry (2004) 15, 1088-1094). Any appropriate antenna molecule may be used for embodiments of the invention. Alternative chelators and energy transfer antenna species are described in Petoud, S., et al., J. Am. Chem. Soc. 2003, 125, 13324-13325 and Parker, D. Coord. Chem. Rev. 2000, 205, 109-130.

In some embodiments, the phosphor component is a species with a lifetime that is 0.1 to 300 μsec, more preferably 1-100 μsec, 10-1000 times shorter than the 1 msec lifetime of Tb+3. This permits a higher excitation repetition rate and thus more rapid data acquisition. As discussed above, the excited-state lifetime of a terbium chelate is on a millisecond time scale. Time-resolved detection techniques on this time scale are easily and inexpensively implemented. The use of an initial 10-100 μs off” gate suppresses all stray light and extraneous fluorescence resulting in extremely low background noise. However, with a 1 msec lifetime, excitation of a terbium luminescence sensor cannot be more frequent than a few hundred times per second. Some transition metal complexes including ruthenium (Ru) and rhenium (Re) have emission lifetimes in the 0.1 to 300 μsec range. (Simon, J. A, et al. J. Am. Chem. Soc. 1997, 119, 11012-11022; Harriman, A, et al. Chem. Commun. 1999, 735-736; Kalayanasundarm, K. Photochemistry of Polypyridine and Porphyrin Complexes; Academic Press: New York, 1992; Juris, A, et al. Coord. Chem. Rev. 1988, 84, 85-277; Tyson, D. S., et al. J. Phys. Chem. A. 1999, 103, 10955-10960; Tyson D S, et al., Inorg. Chem. 40 (16): 4063-4071 (2001); Stufkens, D. J., et al. Pure Appl. Chem. 1997, 69, 831-835; Higgins B, et al., Inorg. Chem. 44 (19), 6662-6669, (2005); Tsubaki H, et al., J. Am. Chem. Soc 127(44), 15544-15555 (2005); Fischer M J, et al., J. Lumin. 114 (1), 60-64 (2005)).

In the case of a species with a 10 μsec lifetime phosphor, it is possible to increase the excitation repetition rate to 10,000/sec. The optical excitations of these complexes are more appropriately termed charge transfer excitations with the excited states being metal to ligand charge transfer states (or ligand to metal charge transfer states). The distinction between a chelator group and a sensitizer, appropriate to the lanthanide embodiments, does not apply for these embodiments. From the point of view of the present invention, the question is whether the interchange rate between the two conformers of the sensing construct is sufficiently rapid to average on the time-scale of the phosphor. If this is the case, then these shorter-lived species provide advantages in certain applications. However, the advantages of this methodology are only realized with more expensive optical excitation devices.

Ligands for the switch include but are not limited to a nucleic acid, protein or other biopolymer, an organism or a small molecule.

Preferably the bistable nucleic acid switch exhibits a binding affinity for the ligand of Kd<1 μM.

Areas of Contemplated Use

(1) Diagnostic tests for the presence of a protein, nucleic acid, supramolecular structure, whole or inactivated organism, or other analyte molecule (A) that binds preferentially to one of the two stable states of S. This stable state contains an analog of a naturally occurring RNA or DNA binding site for A (ligand binding domain).

(2) The discovery of chemical entities (C) that interfere with binding of A to natural RNA or DNA analogs of S. One application involves C molecules that are leads for therapeutic agents against a disease state for which S-L interactions are necessary.

(3) Applications similar to (1), wherein the ligand binding domain of S comprises a combinatorially-derived sequence that is empirically chosen to bind tightly and specifically to A. Embodiments include field kits for real-time detection of infectious organisms or toxic agents.

(4) Applications similar to (2), wherein the ligand binding domain of S comprises a combinatorially-derived sequence that is empirically chosen to bind tightly and specifically to A. Embodiments include the discovery of chemical agents, C, for the remediation of effects due to infectious or toxic agents, A.

(5) Molecular electronic applications where the state change in S occurs in response to a triggering impulse, which may be a light pulse that alters the state of a photosensitive ligand, L1, to L2. In these applications, the ligand binding domain of S may contain a natural RNA or DNA binding site for L1 or L2, or a combinatorially-derived sequence empirically chosen to bind tightly and specifically to either L1 or L2. The shape and properties of S will depend upon whether the combinatorially-derived sequence-binding pocket is occupied. Here, the construct may include a fluorophore quencher pair or other signal generating elements.

The bistable nucleic acid switch may be designed to bind to ligands selected from the group consisting of NC, tat, and rev proteins from HIV-1. or, the ligand binding domain may be adapted to bind a ligand involved in the etiology of a viral infection which is selected from the group consisting of Hepatitis C, Congo-Crimean hemorrhagic fever, Ebola hemorrhagic fever, Herpes, human cytomegalovirus, human pappiloma virus, influenza, Marburg, Q fever, Rift valley fever, Smallpox, Venezuelan equine encephalitis, HIV-1, MMTV, HIV-2, HTLV-1, SNV, BIV, BLV, EIAV, FIV, MMPV, Mo-MLV, Mo-MSV, M-PMV, RSV, SIV, AMV.

In another variation, the ligand binding domain may be adapted to bind a ligand selected from the group consisting of TAR-tat, RRE-rev, DIS, PBS, RT, PR, IN, SU, TM, vpu, vif, vpr, nef, mos, tax, rex, sag, v-src, v-myc and precursors and protease products of the precursors, gag, gag-pol, env, src, and onc as collected in Appendix 2 of (Coffin, J. M., Hughes, S. H., Varmus, H. E. (1997) Retroviruses, Cold Spring Harbor Lab Press, Plainview, N.Y.).

In another variation, the ligand binding domain may be adapted to bind a ligand derived from an organism selected from the group consisting of bacteria, fungi, insects, and pathogens and pests to humans, animals, and plants. Further, the ligand binding domain may be adapted to bind a toxin or other factor derived from bacteria and other microorganisms selected from the group consisting of B. anthracis, Burkholderia pseudomallei, Botulinum, Brucellosis, Candida albicans, Cholera, Clostridium perfringins, Kinetoplasts, Malaria, Mycobacteria, Plague, Pneumocystis, Schistosomal parasites, Cryptosporidium, Giardia, and other environmental contaminants of public and private water supplies, Ricin, Saxitoxin, Shiga Toxin from certain strains of E. coli, Staphylococcus (including enterotoxin B), Trichothecene mycotoxins, Tularemia, and agents causing Toxoplasmosis, as well as contaminants of food and beverages that may be deleterious to human or animal health.

In another embodiment, the ligand binding domain may be adapted to bind a small-molecule target selected from the group consisting of nerve gas agents and chemical poisons, as well as contaminants of public and private water supplies, of food and beverages, and of indoor air that may be deleterious to human or animal health.

In another preferred embodiment of the present invention, a diagnostic method is disclosed for detecting the presence of a ligand molecule in a sample. The diagnostic method comprises the steps of: (1) providing a molecular switch as described above; (2) contacting the molecular switch with the sample; and (3) monitoring changes in the fluorescent signal.

In a preferred variation to the diagnostic method, the molecular switch comprises a chimeric DNA-RNA molecule. The molecular framework may comprise DNA, and the ligand binding domain may comprise RNA. This does not exclude the possibility of the ligand binding domain or molecular framework being composed of either RNA or DNA, nor does it exclude the possibility of one or more monomers in the chain being composed of a modified nucleotide. In one embodiment, the ligand binding domain may comprise a combinatorially-derived sequence which has been empirically chosen to bind said ligand. Preferably, the combinatorially-derived sequence has an affinity for the ligand of at least Kd<1 μM.

The diagnostic method may be adapted to detect ligands selected from an infectious organism or toxic agent. In one mode, the diagnostic method may be adapted for use in a field kit for real-time detection of infectious organisms or toxic agents.

In another preferred embodiment of the present invention, an assay method is disclosed for discovering a chemical entity that interferes with a natural RNA or DNA for binding of a ligand. The assay method comprises the steps of: (1) providing a molecular switch as described above; (2) contacting the molecular switch with the ligand in the absence of the chemical entity, and monitoring the fluorescent signal; (3) contacting the molecular switch with the ligand in the presence of the chemical entity, and monitoring the fluorescent signal; and (4) comparing the fluorescent signals generated in the presence and absence of the chemical entity to determine whether the chemical entity altered the amount of ligand bound to the ligand binding domain.

The molecular switch used in the assay method preferably comprises a chimeric DNA-RNA molecule, wherein the ligand binding domain comprises RNA, the molecular framework comprises DNA, and the ligand is a viral protein. This does not exclude the possibility of the ligand binding domain or molecular framework being composed of either RNA or DNA, nor does it exclude the possibility of one or more monomers in the chain being composed of a modified nucleotide.

In one variation to the assay method, the step of contacting the molecular switch with the ligand in the presence of the chemical entity, further comprises allowing the molecular switch and the ligand to equilibrate prior to adding the chemical entity. Preferably, the molecular switch is adapted to generate a null luminescent signal upon equilibration with the ligand.

In another variation to the assay method, the ligand binding domain may comprise a combinatorially-derived sequence which has been empirically chosen to bind said ligand.

Other Target Interactions

In development of the chimeric switches of the present invention, any other target interactions with RNA, DNA, proteins, precursors, and saccharides may be exploited in accordance with the present disclosure. Some of these targets include, without limitation, the internal ribosome entry site (IRES) of Hepatitis C Virus, IRES sites in other viruses, as well as agents involved in the etiology of viral infections related to Congo-Crimean hemorrhagic fever, Ebola hemorrhagic fever, Herpes, human cytomegalovirus, human pappiloma virus, influenza, Marburg, Q fever, Rift valley fever, Smallpox, Venezuelan equine encephalitis, and targets in HIV-1, MMTV, HIV-2, HTLV-1, SNV, BIV, BLV, EIAV, FIV, MMPV, Mo-MLV, Mo-MSV, M-PMV, RSV, SIV, AMV, and other related retroviruses, including but not limited to: TAR-tat, RRE-rev, DIS, PBS, RT, PR, IN, SU, TM, vpu, vif, vpr, nef, mos, tax, rex, sag, v-src, v-myc and precursors and protease products of the precursors: gag, gag-pol, env, src, onc, as collected in Appendix 2 of (Coffin, J. M., Hughes, S. H., Varmus, H. E. (1997) Retroviruses, Cold Spring Harbor Lab Press, Plainview, N.Y.). Other targets in bacteria, fungi, insects, and other pathogens and pests of humans, animals, and plants may also be applicable to the present switches and methods, including but not limited to B. anthracis, (especially the components of the toxin: protective antigen, lethal factor, edema factor, and their precursors), Burkholderia pseudomallei, Botulinum toxins, Brucellosis, Candida albicans, Cholera, Clostridium perfringins toxins, Kinetoplasts, Malaria, Mycobacteria, Plague, Pneumocystis, Schistosomal parasites, Cryptosporidium, Giardia, and other environmental contaminants of public and private water supplies, Ricin, Saxitoxin, Shiga Toxin from certain strains of E. coli, Staphylococcus (including enterotoxin B), Trichothecene mycotoxins, Tularemia, and agents causing Toxoplasmosis, as well as contaminants of food and beverages that may be deleterious to human or animal health. The detection and screening methodologies afforded by some embodiments of this invention may also be applied to small-molecule targets, including but not limited to nerve gas agents and chemical poisons, as well as contaminants of public and private water supplies, of food and beverages, and of indoor air that may be deleterious to human or animal health.

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A molecular switch, comprising a binding domain for a ligand, a framework and a signaling apparatus, wherein said signaling apparatus comprises a long-lived emitter molecule and short range quencher molecule located along said framework and having changeable positions relative to one another, such that a difference is detectable in a fluorescent signal upon change in conformation between two predominantly populated conformational states of said switch, wherein one conformational state binds the ligand, and wherein there is interchange between these two conformational states that is rapid compared to the emission lifetime of the long-lived emitter.

2. The molecular switch of claim 1, wherein said switch comprises a nucleic acid.

3. The molecular switch of claim 1, wherein said switch includes one or more modified nucleotide monomers.

4. The molecular switch of claim 2, wherein said nucleic acid comprises a double-hairpin construct.

5. The molecular switch of claim 1, wherein the short range quencher is a quencher based upon electron transfer processes.

6. The molecular switch of claim 5, wherein the quencher is a nitroxide.

7. The molecular switch of claim 6, wherein the nitroxide is TEMPOL or a derivative thereof.

8. The molecular switch of claim 1, wherein the long lived emitter molecule is selected from the group consisting of a lanthanide chelate, a ruthenium chelate and a rhenium chelate.

9. The molecular switch of claim 8, wherein the lanthanide chelate is CS124-DTPA.

10. The molecular switch of claim 1, wherein the long lived emitter has a emission lifetime of 10 μsec to 10 msec.

11. The molecular switch of claim 1, wherein the long lived emitter has an emission lifetime of 0.1 to 300 μsec.

12. The molecular switch of claim 1, wherein the ligand is ricin, cryptosporidium or its oocysts, giardia or its cysts, E. coli, Shiga-like toxin producing E. coli O157:H7 strain, Legionella Pneumophila, or Staphylococcus aureus.

13. The molecular switch of claim 1, wherein said ligand is involved in the etiology of a viral infection, which is selected from the group consisting of Hepatitis C, Congo-Crimean hemorrhagic fever, Ebola hemorrhagic fever, Herpes, human cytomegalovirus, human pappiloma virus, influenza, Marburg, Q fever, Rift valley fever, Smallpox, Venezuelan equine encephalitis, HIV-1, MMTV, HIV-2, HTLV-1, SNV, BIV, BLV, EIAV, FIV, MMPV, Mo-MLV, Mo-MSV, M-PMV, RSV, SIV, and AMV.

14. The molecular switch of claim 1, wherein said ligand is selected from the group consisting of TAR-tat, RRE-rev, DIS, PBS, RT, PR, IN, SU, TM, vpu, vif, vpr, nef, mos, tax, rex, sag, v-src, v-myc and precursors and protease products of the precursors, gag, gag-pol, env, src, and onc.

15. The molecular switch of claim 1, wherein said ligand is derived from an organism selected from the group consisting of bacteria, fungi, insects, and pathogens and pests to humans, animals, and plants.

16. The molecular switch of claim 1, wherein said ligand is a toxin or other factor derived from bacteria and other microorganisms selected from the group consisting of B. anthracis, Burkholderia pseudomallei, Botulinum, Brucellosis, Candida albicans, Cholera, Clostridium perfringins, Kinetoplasts, Malaria, Mycobacteria, Plague, Pneumocystis, Schistosomal parasites, Cryptosporidium, Giardia, and other environmental contaminants of public and private water supplies, Ricin, Saxitoxin, Shiga Toxin from certain strains of E. coli, Staphylococcus (including enterotoxin B), Trichothecene mycotoxins, Tularemia, and agents causing Toxoplasmosis, and food or beverage contaminants that may be deleterious to human or animal health.

17. The molecular switch of claim 1, wherein said ligand is a small-molecule target selected from the group consisting of nerve gas agents, chemical poisons, contaminants of public and private water supplies, food and beverage contaminants, and contaminants of indoor air that may be deleterious to human or animal health.

18. A diagnostic method for detecting the presence of a ligand molecule in a sample, comprising the steps of:
providing the molecular switch according to claim 1;
contacting said molecular switch with said sample;
pulsing the molecular switch with an excitation pulse of an appropriate first wavelength;
delaying measurement of the emission spectra for 0.1 μsec to 1 msec; and
measuring the emission spectra at an appropriate second wavelength to determine the presence of the ligand molecule.

19. The method of claim 18, wherein the excitation pulse is for 1-20 ns.

20. The method of claim 18, wherein the luminophore is CS124-DTPA, and the first wavelength is 340 nm with a 30 nm bandpass.

21. The diagnostic method of claim 18, wherein said switch comprises a chimeric DNA-RNA molecule.

22. The diagnostic method of claim 18, wherein said switch includes one or more modified nucleotide monomers.

23. The diagnostic method of claims 18, wherein said ligand is an infectious organism or toxic agent.

24. The diagnostic method of claim 23, wherein said method is adapted for use in a field kit for real-time detection of said infectious organism or toxic agent.

25. The diagnostic method of claim 18, wherein measurement of the emission spectra is delayed for 10 to 500 μsec.

26. The diagnostic method of claim 18, wherein measurement of the emission spectra is delayed for 0.1 to 10 μsec.

27. An assay method for discovering a chemical entity that interferes with ligand binding, comprising the steps of:
(a) providing the molecular switch according to claim 1;
(b) contacting said molecular switch with said ligand in the absence of the chemical entity;
(c) pulsing the molecular switch with an excitation pulse of an appropriate first wavelength;
(d) delaying measurement of the emission spectra for 0.1 μsec to 1 msec;
(e) measuring the emission spectra at an appropriate second wavelength to determine the presence of the ligand molecule, and monitoring the signal;
(f) contacting said molecular switch with said ligand in the presence of the chemical entity;
(g) repeating steps (c)-(e) to determine the binding of the ligand in the presence of the chemical entity; and
(h) comparing the signals generated in the presence and absence of the chemical entity to determine whether the chemical entity interfered with the binding of said ligand.

28. The assay method of claim 27, wherein said switch includes one or more modified nucleotide monomers.

29. The assay method of claim 27, wherein said ligand is a viral protein.

30. The assay method of claim 27, wherein the step of contacting said molecular switch with said ligand in the presence of the chemical entity, further comprises allowing said molecular switch and said ligand to equilibrate prior to adding the chemical entity.

31. The assay method of claim 30, wherein said molecular switch is adapted to generate a null fluorescent signal upon equilibration with said ligand.

32. The assay method of claim 27, wherein said binding domain comprises a combinatorially-derived sequence which has been empirically chosen to bind said ligand.

33. The assay method of claim 27, wherein measurement of the emission spectra is delayed for 10 to 500 μsec.

34. The assay method of claim 27, wherein measurement of the emission spectra is delayed for 0.1 to 10 μsec.
